(19) United States
(12) Patent Application Publication
(10) Pub. No.: US 2020/0069374 A1
(43) Pub. Date: Mar. 5, 2020

(54) SURGICAL SUPPORT DEVICE AND SURGICAL NAVIGATION SYSTEM

(71) Applicant: Hitachi, Ltd., Tokyo (JP)

(72) Inventors: Yusuke SEKI, Tokyo (JP); Nobutaka ABE, Tokyo (JP); Kitaro YOSHIMITSU, Tokyo (JP)

(73) Assignee: Hitachi, Ltd., Tokyo (JP)

(21) Appl. No.: 16/385,079

(22) Filed: Apr. 16, 2019

(30) Foreign Application Priority Data
Sep. 3, 2018 (JP) JP2018-164579

(51) Int. Cl. A61B 34/20; A61B 90/00

(52) U.S. Cl. CPC A61B 34/20; A61B 90/39; A61B 34/25; A61B 2034/2065; A61B 90/36

(57) ABSTRACT

To predict the movement and the deformation of an organ before and after an intervention, and improve the accuracy of position information on a surgical instrument or the like to be presented to an operator. Provided is a surgical support device that is provided with a storage device that stores therein, by analyzing data before and after an intervention to an object, an artificial intelligence algorithm having learned a deformational rule of the object that deforms due to the intervention, and a prediction data generation unit that generates, using the artificial intelligence algorithm, prediction data in which a shape of the object after the intervention is predicted on the basis of the data before the intervention.

### BACKGROUND OF THE INVENTION

### Field of the Invention

The invention relates to a support device for surgery and a navigation system for surgery that support an operator at the surgery using medical images.

### Background Art

Navigation systems for surgery have been known in which treatment plan data created before surgery and data acquired during the surgery are integrated to guide a position and a posture of a surgical instrument or the like, thereby supporting an operator so as to perform the surgery in safety and in security.

In more details, the navigation system for surgery is, for example, such a system that position information in an actual space on various kinds of medical treatment equipment such as a surgical instrument, which is detected by a sensor such as a position measurement device, is displayed superimposed on a medical image preoperatively acquired by a medical image photographing device such as a CT or an MRI, thereby presenting a position of the surgical instrument and supporting the surgery, with respect to an operator.

Meanwhile, there is a case where an organ may move or deform intraoperatively, and in such a case, shapes and positions of a target organ in a medical image preoperatively picked up and a target organ deformed intraoperatively do not necessarily match each other. Accordingly, in the surgical navigation system, even when position information on a surgical instrument is displayed on an image preoperatively photographed, the reliability of the position information may be low in some cases.

As one example of a phenomenon in which such an organ moves and deforms, “brain shift” in which a brain moves and deforms at craniotomy in neurosurgical surgery has been known. Because the brain shift is accompanied by the movement or the deformation of the brain of several millimeters to several centimeters, the accuracy of a surgery navigation based on a preoperative image, in other words, presentation of position information on a surgical instrument or the like is apparently lowered (Non-Patent Literature 1).

Here, the brain shift will be described.

FIGS. 11A and 11B illustrate an example of medical images indicating that a brain shift occurs between before and after the intervention when an operation of removing a brain tumor is performed, in other words, of brain tomographic images before and after the intervention. Specifically, FIG. 11A illustrates the brain tomographic image before the intervention, in other words, before the craniotomy, and FIG. 11B illustrates the brain tomographic image after the intervention, in other words, after the craniotomy.

In the brain tomographic image before the craniotomy illustrated in FIG. 11A, brain tissue 103 and a brain tumor 104 are present in a region surrounded by a skull 102. Here, when surgery of removing the brain tumor 104 is conducted, parts of the skull 102 and dura mater are cut off to form a craniotomy range 106. At this time, as illustrated in FIG. 11B, the brain tissue 103 and the brain tumor 104 that have been floating in the cerebrospinal fluid move and deform from positions before the craniotomy due to the influences of gravity and the like. This phenomenon is called brain shift.

As described the above, in a surgery navigation, conducted is a surgery support of presenting position information on a surgical instrument using a medical image before the surgery in FIG. 11A. However, the brain is in a status as illustrated in FIG. 11B because the brain shift occurs intraoperatively. In other words, a difference is generated between a brain position in a preoperative medical image that is used in the surgical navigation system and an intraoperative brain position, which becomes a factor to lower the accuracy of position information to be presented.

In addition to this, organs may be deformed by the laparotomy surgery, not limited to the neruosurgery. Moreover, the deformation of the organ can occur due to a change in a position of a subject, such as a supine position, a prone position, a standing position, and a sitting position. Therefore, it is considered that predicting the movement and the deformation of an organ before and after an intervention such as surgery and applying them to a surgical navigation system can improve the accuracy of presentation of position information. As a method of predicting the movement and the deformation of an organ, a study related to a structure analysis using a finite element method has been conducted.

### CITATION LIST

### Non Patent Literature

[Non Patent Literature 1] Gerard I J, et al., “Brain shift in neuronavigation of brain tumors: A review “Med Image Anal. 2017 January; 35: 403-420

### SUMMARY OF THE INVENTION

However, as described in Non Patent Literature 1, because the movement and the deformation of an organ between before and after the intervention are complicated phenomena in which a plurality of factors are involved, an accurate physical model is difficult to be constructed, and a practical technique of predicting the movement and the deformation of an organ has not been developed yet. Accordingly, it is difficult to say that position information on a surgical instrument or the like is presented with sufficient reliability in the surgical navigation system.

The invention is made in view of the abovementioned circumstances, and aims to predict the movement and the deformation of an organ between before and after the intervention, and improve the accuracy of position information on a surgical instrument or the like to be presented to an operator.

In order to solve the abovementioned problem, the invention provides the following aspects.

One aspect of the invention provides a surgical support device that is provided with a storage device that stores therein, by analyzing data before and after an intervention to an object, an artificial intelligence algorithm having learned a deformational rule of the object that deforms due to the intervention, and a prediction data generation unit that generates, using the artificial intelligence algorithm, prediction data in which a shape of the object after the intervention is predicted on the basis of the data before the intervention.

Moreover, another aspect of the invention provides a surgical navigation system that is provided with the abovementioned surgical support device.

### Advantage of the Invention

With the invention, it is possible to predict the movement and the deformation of an organ between before and after the intervention, and improve the accuracy of position information on a surgical instrument or the like to be presented to an operator.

### BRIEF DESCRIPTION OF THE DRAWINGS

FIG. 1 is a block diagram illustrating a schematic configuration of a surgical navigation system to which a surgical support device according to an embodiment of the invention is applied;

FIG. 2 is an explanation diagram illustrating a schematic configuration of a position measurement device in the surgical navigation system of FIG. 1;

FIG. 3 is a flowchart for explaining a process to generate an artificial intelligence algorithm related to a surgery support to be applied to the surgical support device according to the embodiment of the invention;

FIG. 4 is a flowchart for explaining a process to generate a prediction medical image serving as prediction data in the surgical support device according to the embodiment of the invention;

FIG. 5 is a flowchart for explaining a process to perform surgery navigation in the surgical navigation system according to the embodiment of the invention;

FIGS. 6A and 6B are explanation diagrams illustrating examples of display images to be displayed on a display of the surgical navigation system according to the embodiment of the invention, FIG. 6A illustrates a preoperative image and a brain shift prediction image, and FIG. 6B illustrates images obtained by superimposing position information on a surgical instrument on the images of FIG. 6A;

FIG. 7 is an explanation diagram illustrating one example of a display screen to be displayed on the display of the surgical navigation system according to the embodiment of the invention;

FIG. 8 is an explanation diagram illustrating one example of a display screen to be displayed on the display of the surgical navigation system according to the embodiment of the invention;

FIG. 9 is a flowchart for explaining a correction process for a prediction medical image in a surgical navigation system according to a first modification example in the embodiment of the invention;

FIG. 10 is a flowchart for explaining a correction process for a prediction medical image in a surgical navigation system according to a second modification example in the embodiment of the invention; and

FIGS. 11A and 11B are reference diagrams of brain tomographic images for explaining a brain shift, FIG. 11A illustrates the brain tomographic image before craniotomy, and FIG. 11B illustrates the brain tomographic image after the craniotomy.

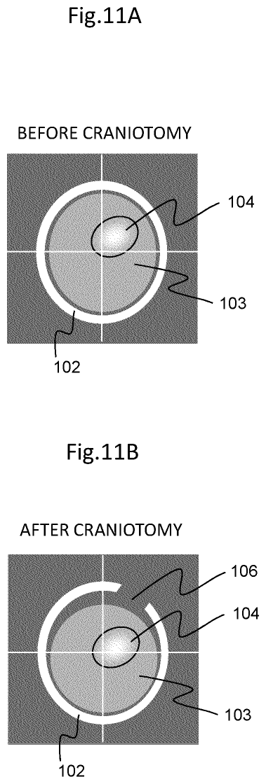

### DETAILED DESCRIPTION OF THE INVENTION

A surgical support device according to an embodiment of the invention is provided with a storage device that stores therein, by analyzing data before and after an intervention to an object, an artificial intelligence algorithm having learned a deformational rule of the object that deforms due to the intervention, and a prediction data generation unit that generates, using the artificial intelligence algorithm, prediction data in which a shape of the object after the intervention is predicted on the basis of the data before the intervention.

Hereinafter, the surgical support device according to the embodiment of the invention will be described in details with reference to the drawings. Note that, in the present embodiment, as one example, the abovementioned surgical support device and a surgical navigation system to which this surgical support device is applied will be described.

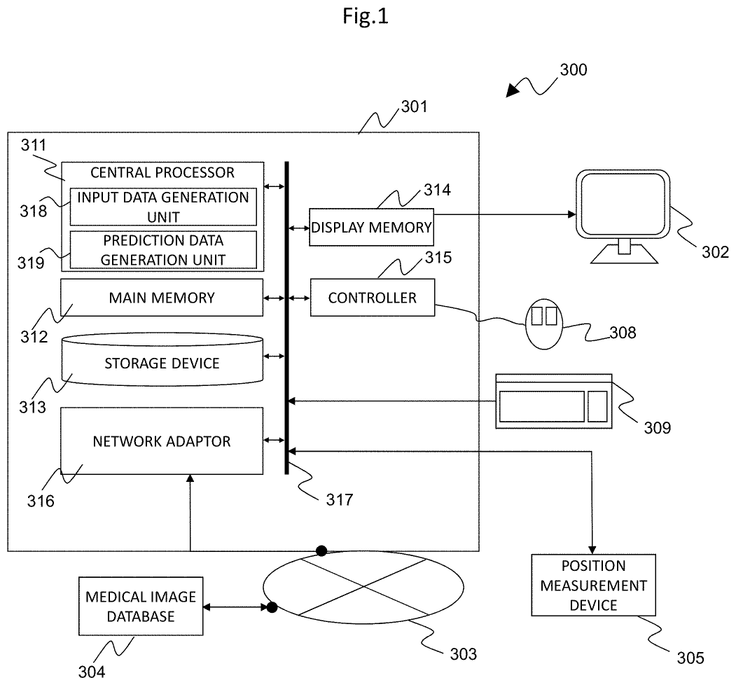

FIG. 1 illustrates a schematic configuration of a surgical navigation system to which the surgical support device according to the embodiment of the invention is applied. This surgical navigation system 300 is provided with a surgical support device 301, a display 302 that displays position information and the like provided from the surgical support device 301, a medical image database 304 that is connected to the surgical support device 301 in a communicable manner via a network 303, and a position measurement device 305 that measures a position of a surgical instrument or the like.

The surgical support device 301 is a device that supports an operator by superimposing position information on a surgical instrument or the like on a desired medical image, and presenting the position information in real time, and is provided with a central processor 311, a main memory 312, a storage device 313, a display memory 314, a controller 315, and a network adaptor 316. These constituents that configure the surgical support device 301 are connected to one another via a system bus 317. Moreover, a key board 309 is connected to the system bus 317 and a mouse 308 is connected to the controller 315, and the mouse 308 and the key board 309 function as an input device that receives an input of a process condition of the medical image.

A general-purpose or dedicated computer that is provided with the abovementioned respective units can be applied as the surgical support device 301. The mouse 308 may be, for example, another pointing device such as a trackpad or a trackball, or the function of the mouse 308 or the key board 309 can be replaced with a display 302, which is caused to have a touch panel function.

The central processor 311 entirely controls the surgical support device 301, and executes a prescribed computation process with respect to a medical image or position information measured by the position measurement device 305, in accordance with a process condition input with the mouse 308 or the key board 309.

Accordingly, as illustrated in FIG. 1, the central processor 311 implements functions of an input data generation unit 318 and a prediction data generation unit 319. Note that, these functions of the input data generation unit 318 and the prediction data generation unit 319 that are implemented by the central processor 311 can be implemented as software in such a manner that the central processor 311 reads and executes a program stored in advance in a memory such as the storage device 313.

Note that, the central processor 311 can be configured by a central processing unit (CPU), a graphics processing unit (GPU), or a combination of the both units. Moreover, a part or all of the operations that are executed by the respective units included in the central processor 311 can be implemented by an application specific integrated circuit (ASIC) or a field-programmable gate array (FPGA).

The input data generation unit 318 generates input data indicating information that is related to an object and includes a shape of the object before the intervention, on the basis of a process condition input by the input device such as the mouse or a medical image read from the medical image database 304. The input data is information necessary for generating prediction data in which a shape of the object after the intervention is predicted. As the input data, in addition to a two-dimensional or three-dimensional medical image read from the medical image database 304, a segmentation image in which a feature region of the object is extracted from the medical image by the image process or a feature point or the like extracted by the image process can be used. When the object is a brain, for example, a skull, brain tissue, a brain tumor, and the like can be considered as feature regions, and an arbitrary point that is included in the outline of these feature regions can be set as a feature point.

The prediction data generation unit 319 generates, using an artificial intelligence algorithm stored in the storage device 313, prediction data in which a shape of the object after the intervention is predicted from the data before the intervention based on the input data generated by the input data generation unit 318. Details of generation of the artificial intelligence algorithm and the prediction data will be described later.

The main memory 312 stores therein a program executed by the central processor 311 and the progress of a computation process.

The storage device 313 analyzes data before and after the intervention on the object, thereby storing therein an artificial intelligence algorithm having learned a deformational rule of the object that deforms due to the intervention. In other words, the storage device 313 analyzes, with respect to an object such as an organ that deforms due to an intervention including a some sort of procedure or surgery, data on shapes and positions before the intervention and after the intervention and a method and a condition of the intervention, thereby generating in advance and storing therein an artificial intelligence algorithm that is related to a surgery support and that has learned the deformational rule of the organ or the like serving as the object.

Moreover, the storage device 313 stores therein a program executed by the central processor 311 and data necessary for the execution of the program. In addition, the storage device 313 stores therein a medical image read from the medical image database 304 and relevant medical treatment information related to the medical image. Examples of the relevant medical treatment information related to the medical image include a diagnostic name, an age, a gender, a position when the medical image is picked up, a position and a posture in the surgery, a tumor site, a tumor region, a craniotomy region, a laparotomy region, an object of a histopathological diagnosis or the like, and surgery relevant information on a target organ. As the storage device 313, for example, a device such as a hard disk capable of transferring data with a portable recording medium such as a CD/DVD, a USB memory, or an SD card.

The display memory 314 temporarily stores therein display data that is used for causing the display 302 to display an image and the like.

The controller 315 detects a state of the mouse 308, acquires a position of a mouse pointer on the display 302, outputs the acquired position information and the like to the central processor 311. The network adaptor 316 connects the surgical support device 301 to the network 303 configured by a local area network (LAN), telephone lines, the Internet, and the like.

The display 302 displays a medical image on which position information generated by the surgical support device 301 is superimposed, thereby providing the medical image and the position information on the surgical instrument or the like to the operator.

The medical image database 304 stores therein a medical image such as a tomographic image of a subject 601 and relevant medical treatment information related to the medical image. A medical image photographed by a medical image photographing device such as an MRI device, an X-ray CT device, an ultrasound imaging device, a scintillation camera device, a PET device, or an SPECT device is preferably stored in the medical image database 304.

Moreover, as the relevant medical treatment information, for example, when the intervention is craniotomy surgery, information, such as a craniotomy region, a position of a patient, and patient information, related to the factors that can be considered to affect the movement and the deformation of an organ is caused to be stored. The medical image database 304 is connected to the network adaptor 316 so as to be capable of transmitting and receiving signals, via the network 303. Here, “so as to be capable of transmitting and receiving signals” indicates a state in which signals are electrically or optically transmittable and receivable mutually or from one to another, regardless of a wired or wireless manner.

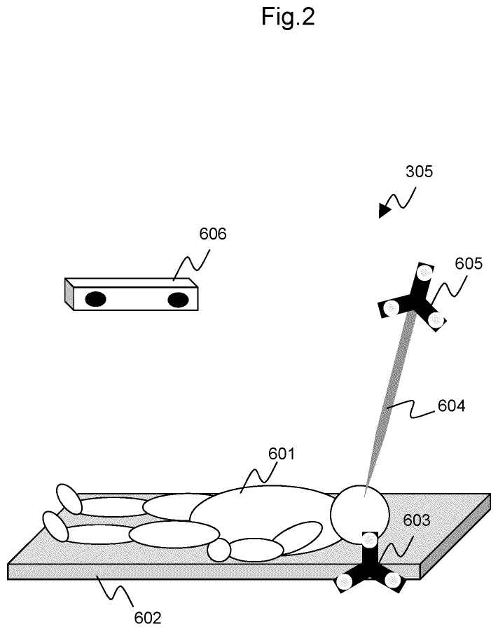

The position measurement device 305 measures a three-dimensional position of a surgical instrument or the like in the subject, after the intervention such as the surgery. In other words, as illustrated in FIG. 2, as for the subject 601 on a bed 602, the position measurement device 305 acquires, by measuring light from a marker 603 provided in the vicinity of the subject 601 and light from a marker 605 provided to a surgical instrument 604 by an infrared ray camera 606, position information indicating a position of the surgical instrument 604 or the like in the subject 601, and outputs the acquired position information to the central processor 311 via a system bus 317.

(Generation of Artificial Intelligence Algorithm Related to Surgery Support)

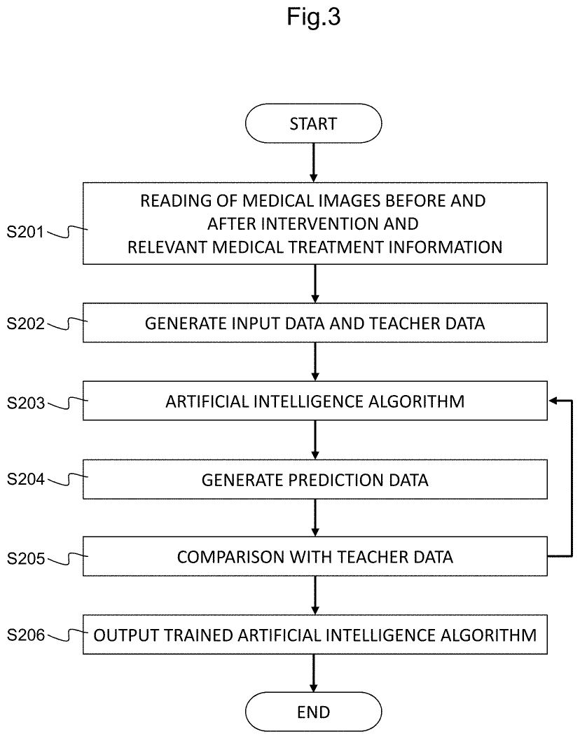

As mentioned the above, the storage device 313 stores therein an artificial intelligence algorithm having learned a deformational rule of the object that deforms due to the intervention. This artificial intelligence algorithm related to a surgery support is a trained artificial intelligence algorithm that analyzes data such as shapes and positions before the intervention and after the intervention, and a method and a condition of the intervention, with respect to an object such as an organ that deforms due to an intervention including a some sort of procedure, surgery, or the like, and has learned a deformational rule of the organ, serving as the object. The artificial intelligence algorithm is generated in accordance with a procedure indicated in a flowchart illustrated in FIG. 3, for example.

The artificial intelligence algorithm can be generated using a surgical support device, and can be also generated using another computer or the like. In the following explanation, an example in which an artificial intelligence algorithm having learned the prediction of a brain shift, in other words, a deformational rule of a brain shape between before and after the intervention, is generated using a surgical support device will be described.

As illustrated in FIG. 3, at Step S201, the central processor 311 performs reading of medical images before and after the intervention, in other words, before and after the craniotomy, and relevant medical treatment information related to the medical images, from the medical image database 304. At Step S202, on the basis of the medical images and the relevant medical treatment information read at Step S201, input data and teacher data for being input to the artificial intelligence algorithm and for causing the artificial intelligence algorithm to learn are generated.

As for the input data to be generated related the medical images, as mentioned the above, in addition to the medical image, a segmentation image in which a feature region is extracted from the medical image, and a feature point can be used as the input data. Similarly, as teacher data, in addition to the medical image, the segmentation image, and the feature point, a displacement matrix that associates movements and deformations of the object before and after the intervention with each other can be used.

At subsequent Step S203, Step S204, and Step S205 are processes of a machine learning by the artificial intelligence algorithm. The input data is substituted into the artificial intelligence algorithm at Step S203, prediction data is acquired at Step S204, and the acquired prediction data is compared with teacher data at Step S205. Further, a comparison result is fed back to the artificial intelligence algorithm and corrected, in other words, the processes from Step S203 to Step S205 are repeated, thereby optimizing the artificial intelligence algorithm such that an error between the prediction data and the teacher data becomes the minimum.

As for the artificial intelligence algorithm, for example, an artificial intelligence algorithm of a Deep learning such as a Convolutional neural network is preferably used, but is not limited thereto. At Step S205, if a desired condition is satisfied, a trained artificial intelligence algorithm is output at Step S206.

The optimized artificial intelligence algorithm is also called a trained network, and is a program having an ability similar to a function to output specific data to the input data. The artificial intelligence algorithm that is optimized in the present embodiment is an artificial intelligence algorithm related to a surgery support that outputs prediction data on a brain shape after the intervention with respect to data before the intervention. The trained artificial intelligence algorithm related to the surgery support generated (optimized) in this manner is stored in the storage device 313.

Note that, examples of prediction data to be generated at Step S204 include a medical image of an organ after having moved and deformed, a segmented medical image, a value quantitatively indicating the amount of movement or the amount of deformation, or a displacement field matrix that associates an image after the intervention with an image before the intervention.

Further, using the trained artificial intelligence algorithm related to the surgery support stored in the storage device 313, the surgical navigation system 300 configured as mentioned the above generates prediction data on an organ of an intervention target on the basis of input data, and conducts a surgery navigation that presents position information on a surgical instrument or the like with respect to an operator, using the prediction data.

(Generation of Prediction Data Using Artificial Intelligence Algorithm Related to Surgery Support)

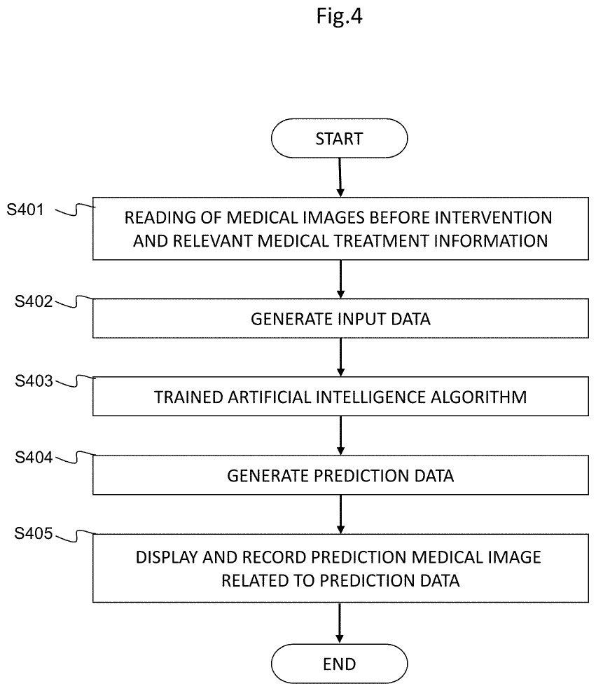

Hereinafter, a process to generate prediction data using the artificial intelligence algorithm in the surgical support device according to the present embodiment will be described in accordance with a flowchart of FIG. 4. Note that, prediction data to be generated in the present embodiment is a medical image indicating a brain deformation after the intervention, in other words, after the craniotomy.

At Step S401, from the medical image database 304, reading of a medical image having been photographed before the intervention, in other words, before the craniotomy, and relevant medical treatment information is performed. At Step S402, on the basis of the data read at Step S401, the input data generation unit 318 generates input data for being input to the artificial intelligence algorithm stored in the storage device 313.

At Step S403, the input data is substituted into the artificial intelligence algorithm, and the prediction data generation unit 319 performs a computation in accordance with the artificial intelligence algorithm, and generates prediction data at Step S404. The central processor 311 causes the acquired prediction data to be stored in the storage device 313 or the medical image database 304, and to be displayed on the display 302 via the display memory 314 at subsequent Step S405.

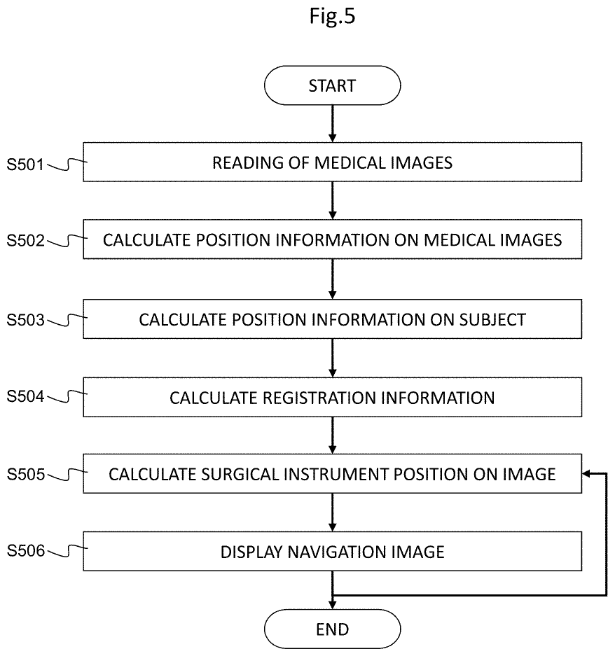

Subsequently, a surgery navigation process (position information presentation process) in the surgical navigation system 300 according to the present embodiment will be described in accordance with a flowchart of FIG. 5.

Position information that is used in the following explanation includes information on a position, a direction, a posture, and the like in space coordinates of a measurement target.

At Step S501, the central processor 311 acquires, from the medical image database 304, a medical image that is preoperatively picked up and a prediction medical image indicating a shape of the object after the intervention generated by the prediction data generation unit 319 as prediction data. At Step S502, from Digital Imaging and Communication in Medicine (DICOM) information on the acquired medical image, relevant medical treatment information, and information related to the prediction medical image, positions and directions of the medical image and the prediction medical image that are used for navigation are acquired.

At Step S503 the central processor 311 uses the position measurement device 305 to measure position information on the marker 603 for detecting a position of the subject 601 (see FIG. 2). At Step S504, from position information on the medical image and position information on the subject acquired at Step S502 and at Step S503, a position on the medical image corresponding to the subject position is calculated, and alignment of the position of the subject with the position on the medical image is performed.

At Step S505, position information on a surgical instrument is measured so as to guide an operation of the surgical instrument or the like. In other words, the central processor 311 uses the position measurement device 305 to measure position information on the marker 605 provided to the surgical instrument 604, and calculates coordinates of the marker 605 in a medical image coordinate system. Note that, the position information on the marker 605 includes an offset to a distal end of the surgical instrument 604.

At Step S506, a navigation image in which the position information on the surgical instrument or the like acquired at Step S505 is superimposed on the medical image, and is caused to be displayed on the display 302 via the display memory 314. In this process, as for a medical image that is displayed by position information on the surgical instrument or the like being superimposed thereon, either one or both of a medical image before the intervention that is preoperatively acquired or a medical image after the intervention that is generated by the prediction data generation unit can be selected.

Note that, an image process condition (parameter and the like) can be input by the mouse 308 and the key board 309, and the central processor 311 generates a navigation image that is subjected to a process in accordance with the input image process condition.

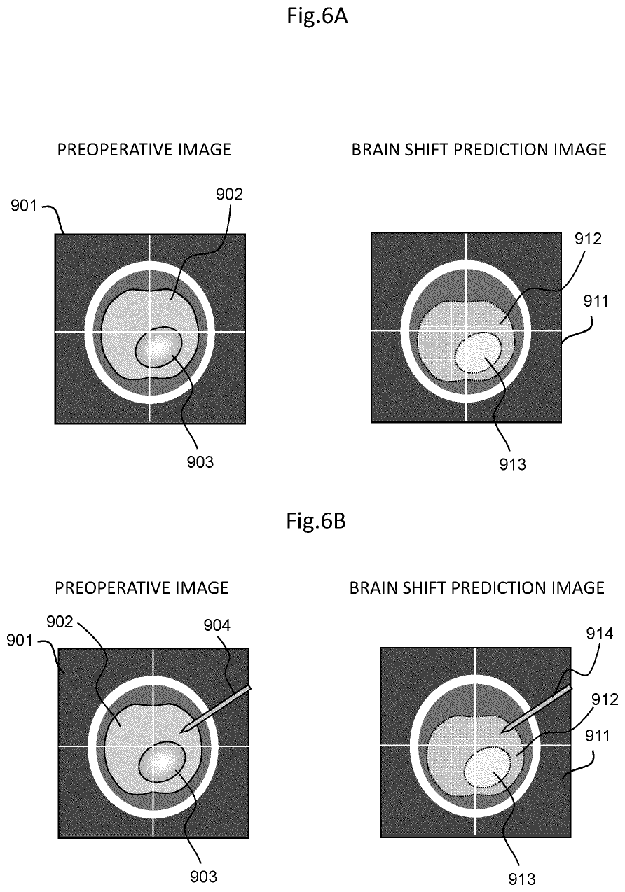

FIGS. 6A and 6B illustrate examples of a display screen on which a navigation image generated by the surgical navigation system according to the present embodiment is displayed on the display 302. FIG. 6A illustrates the example in which a medical image before the intervention, in other words, a preoperative image 901 and a prediction medical image after the intervention, in other words, a brain shift prediction image 911 are simultaneously displayed, and FIG. 6B illustrates the example in which position information on a surgical instrument 914 is displayed superimposed on the medical image of FIG. 6A.

In the preoperative image 901 of FIG. 6A, a segmented brain tissue 902 before the craniotomy and a brain tumor 903 before the craniotomy are displayed. In the brain shift prediction image 911 of FIG. 6A, a predicted brain tissue 912 after the craniotomy and a predicted brain tumor 913 after the craniotomy are displayed.

As illustrated in FIG. 6B, position information on a surgical instrument is caused to be displayed on both of the preoperative image 901 and the brain shift prediction image 911 in FIG. 6A, so that it is possible to simultaneously conduct both of a surgery navigation based on the preoperative image 901 and a surgery navigation based on the brain shift prediction image 911. In FIGS. 6A and 6B, as one example, axial cross-sections are illustrated, however, similarly, for any of sagittal cross-sections, coronal cross-sections, and three-dimensional rendering images, it is possible to simultaneously display the preoperative image and the brain shift prediction image.

With the present embodiment as in the forgoing, from position information, which is measured by the position measurement device 605, on the marker 603 that is fixed to the subject 601 or a rigid body such as a bed or a fixture a relative positional relationship of which with the subject 601 does not change, and DICOM information that is image position information assigned to the medical image, alignment information necessary for alignment of the subject position with the image position is generated. Further, a navigation image in which the position information on the surgical instrument 604 acquired from the marker 605 that is provided to the surgical instrument 604 can be virtually superimposed on the medical image is generated, and can be caused to be displayed on the display 302.

In this process, the position information is superimposed on the prediction image after the intervention, whereby it is possible to improve the accuracy of the position information on the surgical instrument or the like to be presented even in a case where the organ moves or deforms after the intervention. In other words, with the surgical navigation system according to the present embodiment, a prediction medical image in which the movement and the deformation of an organ between before and after the intervention is predicted is generated, and is used as a navigation image, thereby making it possible to improve the image guidance of the surgical instrument in a short time with respect to the operator, in other words, the accuracy of the position information on the surgical instrument or the like to be presented to the operator.

### ANOTHER DISPLAY EXAMPLE 1 OF NAVIGATION IMAGE

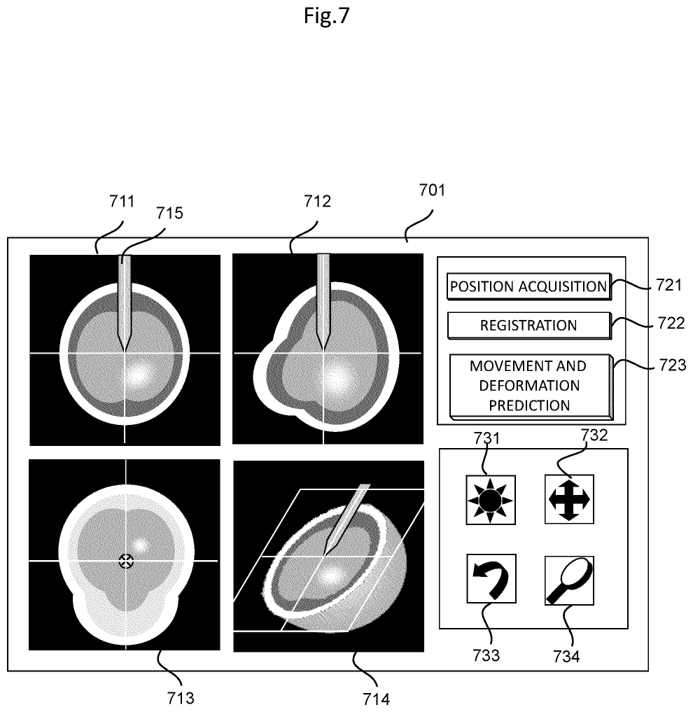

FIG. 7 illustrates another example of a display screen on which a navigation image generated by the surgical navigation system according to the present embodiment is caused to be displayed on the display 302. As illustrated in FIG. 7, on a display screen 701, as navigation images, a virtual surgical instrument display 715 formed in accordance with the alignment information is displayed by being overlapped with an axial cross-section 711, a sagittal cross-section 712, and a coronal cross-section 713, which are orthogonal three cross-sectional images of a surgery part of the subject 601, and a three-dimensional rendering image 714.

Moreover, in an upper-right portion of the display screen 701, a position acquisition icon 721 serving as a subject position measurement interface, a registration icon 722 serving as an alignment execution interface, and a movement and deformation prediction icon 723 that displays a medical image in which the movement and the deformation by the intervention are predicted are displayed for input.

A user presses down the position acquisition icon 721 so that a measurement instruction of a subject position by the position measurement device 305 is accepted, and presses down the registration icon 722 so that a position of the subject on the medical image in accordance with position information on the subject that is measured by the position measurement device 305 is calculated, and alignment of the position of the subject with the position of the medical image is performed. Moreover, the user presses down the movement and deformation prediction icon 723 to cause the generated prediction medical image to be displayed on the display 302.

Moreover, in a lower-right portion of the display screen 701, icons of an image threshold interface 731 with which an image process command is input, a viewpoint position parallel movement interface 732, a viewpoint position rotation movement interface 733, and an image enlargement interface 734 are displayed. The user can adjust a display area of a medical image by operating the image threshold interface 731. Moreover, the user can move in parallel the position of a viewpoint with respect to the medical image by operating the viewpoint position parallel movement interface 732, and can rotate and move the position of the viewpoint by operating the viewpoint position rotation movement interface 733. In addition, the user can enlarge a selection region by operating the image enlargement interface 734.

### ANOTHER DISPLAY EXAMPLE 2 OF NAVIGATION IMAGE

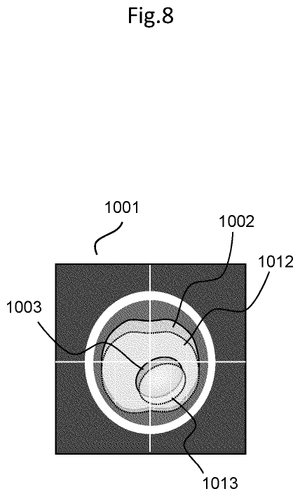

FIG. 8 illustrates another example of a display screen on which a navigation image generated by the surgical navigation system according to the present embodiment is displayed on the display 302. FIG. 8 illustrates an example in which a prediction medical image after the intervention, in other words, a brain shift prediction image is displayed superimposed on a medical image before the intervention, in other words, a preoperative image.

In a superimposed image 1001 of the preoperative image and the brain shift prediction image, a segmented brain tissue 1002 before the craniotomy, a brain tumor 1003 before the craniotomy, a predicted brain tissue 1012 after the craniotomy, and a predicted brain tumor 1013 after the craniotomy are displayed. Here, it is possible to switch between a display of only the preoperative image and a display of only the brain shift prediction image, in accordance with a status.

Moreover, in FIG. 8, as one example, an axial cross-section is illustrated, however, similarly, for any of a sagittal cross-section, a coronal cross-section, and a three-dimensional rendering image, it is possible to display a preoperative image and a brain shift prediction image in a superimposed manner.

### FIRST MODIFICATION EXAMPLE

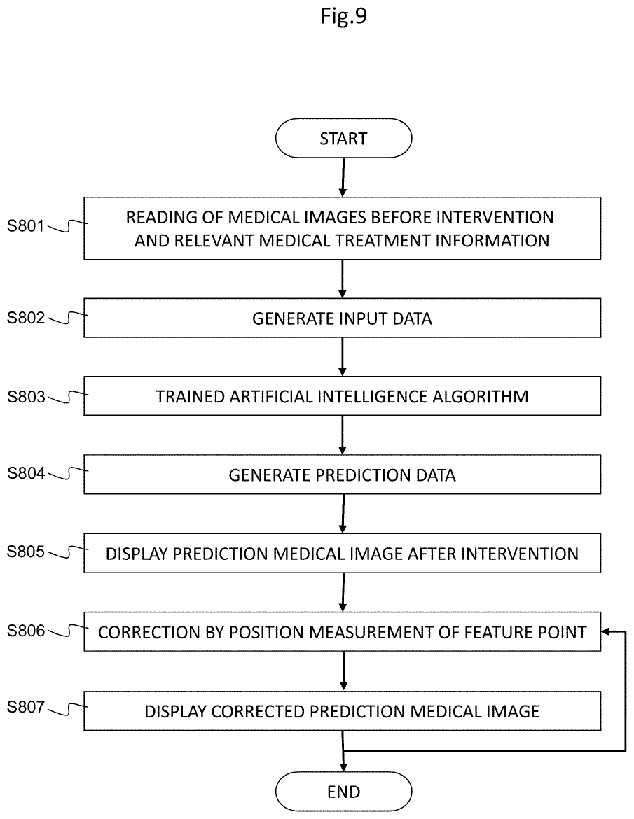

A surgery support navigation system according to a first modification example of the embodiment will be described. In the modification example, a prediction medical image generated using the trained artificial intelligence algorithm related to the surgery support that is stored in the storage device 313 is corrected if necessary. In other words, a prediction medical image serving as prediction data generated by the surgical support device 301 is corrected whenever necessary in such a manner that the surgical instrument 604 measures a position of an organ of the actual subject 601. Hereinafter, a flow of a correction process of a prediction medical image in the modification example will be described in accordance with a flowchart of FIG. 9.

In FIG. 9, at from Step S801 to Step S805, similar to at from Step S401 to S405 in the flowchart of FIG. 4, a process to generate prediction data and cause the prediction data to be displayed on a display is performed. In other words, the central processor 311 performs reading of a medical image having been photographed before the intervention, in other words, before the craniotomy, and relevant medical treatment information from the medical image database 304 at Step S801, and generates input data on the basis of the read data at Step S802.

At Step S803 to at Step S804, the input data is substituted into the artificial intelligence algorithm, and the prediction data generation unit 319 performs a computation in accordance with the artificial intelligence algorithm, and generates prediction data. At subsequent Step S805, the central processor 311 causes the acquired prediction data to be stored in the storage device 313 or the medical image database 304, and to be displayed on the display 302 via the display memory 314.

At Step S806, the central processor 311 uses the position measurement device 305 to measure position information on the surgical instrument 604. In other words, the surgical instrument 604 is used as a probe to measure real position information on a surface of the organ after having moved and deformed by the intervention and a feature point. For example, in a case of neurosurgical surgery, in a state where the prediction medical image is caused to be displayed on the display 302 at Step S805, the distal end of the surgical instrument 604 is disposed on a brain surface, so that it is possible to grasp a position of a real brain surface in which a brain shift occurs by the craniotomy. In this process, when a shift occurs between the brain surface that is displayed on the prediction medical image at Step S805 and the position of the distal end of the surgical instrument 604, the prediction medical image is corrected whenever necessary using the measured feature point.

At Step S807, the corrected prediction medical image is displayed on the display 302. Here, the correction process of the prediction medical image by the position measurement of the feature point can be performed whenever necessary, and each of the medical image before the intervention, the prediction medical image after the intervention, and the corrected prediction medical image can be displayed by a screen operation whenever necessary. Note that, the abovementioned correction process maybe performed with respect to the medical image before the intervention.

### REFERENCE EXAMPLE

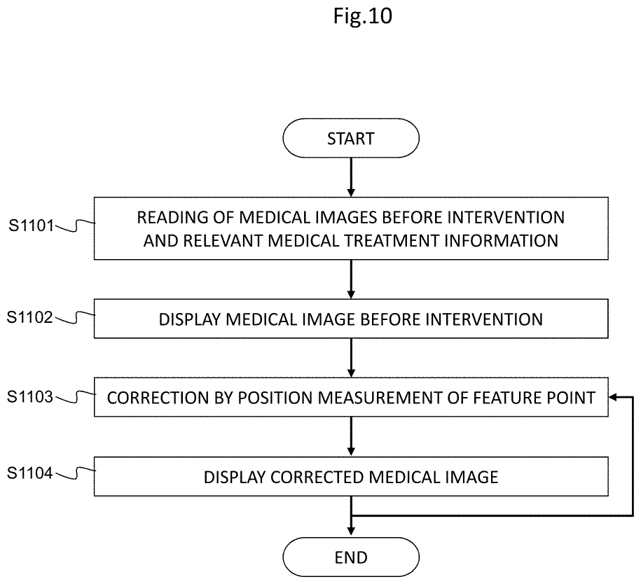

A reference example of a surgery support navigation system according to the present embodiment will be described. In the reference example, in place of the prediction image in which the trained artificial intelligence algorithm is used, a prediction image is generated by intraoperative position information measurement, in other words, a medical image before the intervention is corrected by the position measurement of the feature point. Hereinafter, a correction process according to the reference example will be described in accordance with a flowchart of FIG. 10.

At Step S1101, from the medical image database 304, reading of a medical image before the intervention, in other words, before the craniotomy is performed. At Step S1102, the medical image is displayed on the display 302. At Step S1103, the surgical instrument 604 is used as a probe to measure real position information on a surface of the organ after having moved and deformed by the intervention and a feature point.

More specifically, in a state where the medical image before the intervention is displayed on the display 302 at Step S1102, a feature point is designated on the screen, and a position of the distal end of the surgical instrument 604 is measured by being disposed on the corresponding feature point after the real movement, so that it is possible to define a change in position of feature points before and after the craniotomy.

In addition, an image before the intervention is corrected on the basis of a boundary condition that is defined by designating a feature point a position of which changes before and after the intervention and a feature point that is estimated that a position of which does not change before and after the intervention, so that it is possible to generate and display an image close to that in a state after the intervention. At Step S1104, the corrected medical image is displayed on the display 302. Here, the correction process of the medical image by the position measurement of the feature point can be performed whenever necessary, and the medical image before the intervention and the corrected medical image can be respectively displayed by a screen operation whenever necessary.

With the present embodiment, from data before and after a specified intervention is provided, a trained artificial intelligence algorithm having learned a phenomenon that a subject moves and deforms due to the intervention is generated, and using the trained artificial intelligence algorithm, from image data before the intervention and surgery relevant information, an image of the subject after the intervention is predicted. Further, the predicted image is caused to be displayed on the display, and position information on the subject and a surgical instrument is acquired and superimposed on the medical image, thereby allowing the more sophisticated surgery navigation, compared with the conventional surgery navigation using a preoperative image.

1. A surgical support device comprising:
a storage device that stores therein, by analyzing data before and after an intervention to an object, an artificial intelligence algorithm having learned a deformational rule of the object that deforms due to the intervention; and
a prediction data generation unit that generates, using the artificial intelligence algorithm, prediction data in which a shape of the object after the intervention is predicted on the basis of the data before the intervention.

2. The surgical support device according to claim 1, wherein the data is a medical image and/or surgery relevant information on the object.

3. The surgical support device according to claim 1, wherein the data includes an image of a region indicating the object or a feature point of the object, which are extracted by an image process.

4. The surgical support device according to claim 1, wherein the prediction data includes a displacement matrix that associates the object before the intervention with the object after the intervention.

5. The surgical support device according to claim 1, wherein the intervention is craniotomy surgery, and the object is a brain.

6. The surgical support device according to claim 1, wherein the intervention is laparotomy surgery, and the object is a digestive organ such as a liver.

7. The surgical support device according to claim 1, wherein the intervention is thoracotomy surgery, and the object is a lung or a heart.

8. A surgical navigation system comprising:
the surgical support device according to claim 1;
a display that displays a medical image; and
a position measurement device that measures position information on a subject and a position of a surgical instrument, wherein
the surgical navigation system causes prediction data generated by the surgical support device and the positions of the subject and the surgical instrument measured by the position measurement device to be displayed by being superimposed, on the display.

9. The surgical navigation system according to claim 8, wherein position information on a feature point of the subject is measured by the position measurement device, and the prediction data is corrected using the position information.
